SEQUENCE LISTING (1 sequence)

SEQ ID NO: 1 - protein, 25 aa, synthetic construct
  SITE 1: note = D-Phenylalanine
  SITE 2: note = D-Arginine
  SITE 3: note = D-Tryptophan
  SITE 4..5: note = D-Leucine
  SITE 6: note = D-Arginine
  SITE 7: note = D-Glutamine
  SITE 8: note = D-Leucine
  SITE 9: note = D-Alanine
  SITE 10: note = D-Arginine
  SITE 11: note = D-Leucine
  SITE 12: note = D-Alanine
  SITE 13: note = D-Glutamine
  SITE 14: note = D-Leucine
  SITE 15: note = D-Alanine
  SITE 16: note = D-Lysine
  SITE 17: note = D-Leucine
  SITE 18: note = D-Threonine
  SITE 19: note = D-Proline
  SITE 20: note = D-Valine
  SITE 21: note = D-Lysine
  SITE 22: note = D-Leucine
  SITE 23: note = D-Threonine
  SITE 24: note = D-Proline
  SITE 25: note = D-Valine
FRWLLRQLARLAQLAKLTPVKLTPV

(19) United States
(12) Patent Application Publication
(10) Pub. No.: US 2026/0284139 A1
(43) Pub. Date: Sep. 24, 2026

(54) Administration of a Beta-Catenin Antagonist and Methods of Use

(71) Applicant: Sapience Therapeutics, Inc., Tarrytown, NY (US)

(72) Inventors: Barry Jay Kappel, Pelham, NY (US); Jimmy Andrew Rotolo, Port Washington, NY (US); Gene Merutka, Phoenixville, PA (US); Abi Vainstein Haras, Cresskill, NJ (US); Alice Susannah Bexon, Montclair, NJ (US)

(21) Appl. No.: 19/474,997

(22) PCT Filed: Apr. 13, 2024
(86) PCT No.: PCT/US2024/024529

§ 371 Date: Oct. 13, 2025

Related U.S. Application Data
(60) Provisional application No. 63/459,206, filed on Apr. 13, 2023.

(51) Int. Cl. A61K 38/16; A61K 31/4745; A61K 31/513; A61K 31/517; A61K 31/519; A61P 35/00; C07K 16/22

(52) U.S. Cl. CPC A61K 38/16; A61K 31/4745; A61K 31/513; A61K 31/517; A61K 31/519; A61P 35/00; C07K 16/22

(57) ABSTRACT

Provided are methods of administering a peptide antagonist of β-catenin and methods of treating solid tumors by administering a peptide antagonist of β-catenin.

### BACKGROUND

The Wnt/β-catenin signaling pathway regulates key cellular functions including proliferation, differentiation, migration and stem cell renewal, and plays critical roles in embryonic development and adult tissue homeostasis (Liu 2022). Dysregulation of this pathway often involves constitutive activation of β-catenin. an oncogenic transcription factor implicated in the pathogenesis of many malignancies; it is associated with poor prognosis in many cancers (Shang 2017). Wnt/β-catenin signaling is also associated with promotion of an immunosuppressive tumor microenvironment characterized by decreased chemokine expression, impaired recruitment of dendritic cells, and a subsequent reduction in tumor-infiltrating effector T cells (Ruiz de Galarreta 2019).

In normal human cells, β-catenin cytoplasmic and nuclear expression is tightly regulated by the adenomatous polyposis coli (APC) complex, a protein complex that targets excess β-catenin for proteolytic degradation. Loss-of-function mutations of components of the APC complex or gain-of-function mutations of β-catenin enable β-catenin to escape degradation and accumulate within the nucleus. Nuclear β-catenin engages the transcription factors lymphoid enhancer factor/T cell factor (LEF/TCF) to induce expression of oncogenes that promote cell survival, proliferation, and migration (Bugter 2021).

In cancer cells, accumulation and retention of β-catenin within the nucleus is facilitated by B-cell CLL/lymphoma 9 protein (BCL9) (de la Roche 2008), a co-activator that shuttles β-catenin to the nucleus and acts as a scaffold to recruit the enhancer protein Pygopus to the β-catenin transcription complex to enhance transcriptional activity (Mieszcanek 2019). BCL9 is overexpressed in many tumors and is correlated with poor prognosis (Moor 2015, Vafaizadeh 2021, Wang 2019). Studies have shown that BCL9/BCL9L deletion inhibits tumor growth and proliferation while having only minimal impact upon normal tissue homeostasis, suggesting that β-catenin antagonists may result in significant anti-tumor effect, while unlikely to cause on-target off-tissue toxicity (Gay 2019).

Based on its critical role in promoting malignant phenotypes and the inverse correlation with disease prognosis in several cancer types. β-catenin has been recognized as a potential therapeutic target for cancer therapy (Liu 2022). However, despite extensive ongoing research, β-catenin is conventionally considered to be “undruggable,” and there are no current drugs available to target Wnt/β-catenin signaling.

Previous approaches at targeting the Wnt/β-catenin pathway for cancer therapy act upstream of β-catenin regulation or at the site of β-catenin interaction with LEF/TCF, causing non-specific disruption of the canonical Wnt pathway and resulting in on-target toxicities of the bone (Zhong 2016) and intestine (Dotan 2020). An approach that specifically disrupts the interaction between β-catenin and BCL9 is needed to avoid adverse effects, such as on-target toxicities.

### SUMMARY OF THE INVENTION

Some of the main aspects of the present invention are summarized below. Additional aspects are described in the Detailed Description of the Invention. Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

The disclosure provides a method of treating a solid tumor in a patient, the method comprising parenterally administering to the patient a pharmaceutical composition comprising an effective amount of a peptide antagonist of β-catenin. Also provided is a pharmaceutical composition for parenteral administration comprising an effective amount of a peptide antagonist of β-catenin for use in treating a solid tumor in a patient.

In one embodiment, the solid tumor is selected from the group consisting of breast cancer, colorectal cancer, cholangiocarcinoma, hepatocellular carcinoma, melanoma, non-small cell lung cancer, ovarian cancer, endometrial cancer, pancreatic adenocarcinoma, and synovial sarcoma. In certain embodiments, the patient has received a previous treatment selected from the group consisting of chemotherapy, hormone-based therapy, radiation, targeted therapy, immunotherapy, and combinations thereof. In a particular embodiment, the peptide antagonist is administered as a neoadjuvant as a single agent or in combination. In one embodiment, the peptide antagonist is administered in combination with (i) bevacizumab and/or (ii) folinic acid, fluorouracil, and irinotecan (FOLFIRI). In one embodiment, the peptide antagonist is administered in combination with fruquintinib.

In some embodiments, the peptide antagonist comprises the D-amino acid sequence FRWLLRQLARLAQLAKLTPVKLTPV (SEQ ID NO: 1). In some embodiments, the peptide antagonist is administered to the patient at a dose of about 0.25-12 mg/kg.

One aspect of the invention is a method of administering a peptide antagonist of β-catenin to a subject, the method comprising parenterally administering to the subject a pharmaceutical composition comprising the peptide antagonist, wherein the peptide antagonist comprises the D-amino acid sequence FRWLLRQLARLAQLAKLTPVKLTPV (SEQ ID NO: 1), and wherein the peptide antagonist is administered at a dose of about 0.25-12 mg/kg. Also provided is a pharmaceutical composition comprising a peptide antagonist of β-catenin for use in a method of parenterally administering the peptide antagonist to a subject, wherein the peptide antagonist comprises the D-amino acid sequence FRWLLRQLARLAQLAKLTPVKLTPV (SEQ ID NO: 1), and wherein the peptide antagonist is administered at a dose of about 0.25-12 mg/kg.

In certain methods and compositions of the invention, the peptide antagonist is administered at a dose selected from about 0.5 mg/kg, about 1 mg/kg, about 2 mg/kg, about 4 mg/kg, about 8 mg/kg, and about 12 mg/kg.

In some aspects of the invention, the peptide antagonist disrupts binding of Q-catenin to B-cell CLL/lymphoma 9 protein (BCL9). In certain embodiments, the peptide antagonist comprises an N-terminal octanoyl group. In a particular embodiment, the peptide antagonist is ST316.

In a particular embodiment, the pharmaceutical composition is administered intravenously, for example, via infusion. In embodiments in which the composition is administered via infusion, the infusion duration can be, for example, about 30-180 minutes or about 60-90 minutes.

In some embodiments, the pharmaceutical composition is administered once weekly or once every two weeks. In one embodiment, the pharmaceutical composition is administered for at least four weeks.

### BRIEF DESCRIPTION OF THE DRAWINGS

FIG. 1 shows that ST316 directly binds β-catenin Arm1 with greater affinity than transcription co-activator B-cell CLL/lymphoma 9 protein (BCL9) in a fluorescence polarization assay. Results are plotted as normalized milliP (mP) versus β-catenin concentration.

FIG. 2 shows that ST316 inhibits the protein-protein interaction between β-catenin and BCL9 in an amplified luminescent proximity homogeneous assay (ALPPHA). ST316 (circles) and BCL9 HD2 peptide (squares) both inhibited the interaction of 3-catenin and BCL9 with an IC₅₀ of 1 μM.

FIG. 3A-3B show attenuation of β-catenin nuclear localization by 3 μM or 5 μM ST316 in HCT116 colorectal cancer cells. YY1 and GAPDH were used as markers for the nuclear and cytoplasmic fractions, respectively (FIG. 3A).

FIG. 4A-4D show that ST316 enhances proteasomal degradation of BCL9 in Colo320 colorectal cancer cells.

FIG. 5A-5B show that ST316 inhibits migration of HCT116 colorectal cancer cells across a semi-permeable membrane in a 24-hour invasion assay, compared with a control peptide. FIG. 5A shows cells after treatment with 1 M of a negative control peptide (top panel) or ST316 (bottom panel). FIG. 5B shows cell counts after treatment.

FIG. 6A-6C show that ST316 reduces spontaneous adenoma formation in a murine model of colorectal cancer.

FIG. 7 shows that administration of ST316 (PCAP) inhibits tumor growth in a subcutaneous xenograft tumor model of using HCT116 cells. N=6 mice per group.

FIG. 8A-8B show that administration of ST316 inhibits tumor growth (FIG. 8A) and reduces Axin2 expression (FIG. 8B) in a subcutaneous xenograft tumor model using 4T1-luc triple-negative breast cancer (TNBC) cells. N=6 mice per group.

FIG. 9A-9B show ST316 plasma concentrations over time for Cohorts 1-4 (0.5, 1, 2, or 4 mg/mL).

FIG. 10A-10C show ST316-induced reduction of immune-suppressive polymorphonuclear (PMN) myeloid-derived suppressor cell (MDSC) population in cancer patients. Peripheral blood samples were collected from a patient with pancreatic ductal adenocarcinoma (PDAC) (FIG. 10A) or colorectal cancer (CRC) (FIG. 10B) before (top panels) and after treatment with 4 mg/kg ST316, QW for 3 weeks (bottom panels). Blood was processed to collect lymphocyte and myeloid cells and analyzed by flow cytometry. PMN cells (CD3−, CD19−, CD14−, HLADRlow, CDllb+ and CD15+) were quantified as a percentage of myeloid cells (CD3−, CD19−) (FIG. 10C).

### DETAILED DESCRIPTION OF THE INVENTION

The practice of the present invention can employ, unless otherwise indicated, conventional techniques of pharmaceutics, formulation science, protein chemistry, cell biology, cell culture, molecular biology, microbiology, recombinant DNA, immunology, clinical pharmacology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### I. Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B. A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International d'Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value (i.e., intermediate) encompassed by the range, including integers and fractions. For example, a stated range of 5-10 is also a disclosure of 5, 6, 7, 8, 9, and 10 individually, and of 5.2, 7.5, 8.7, and so forth.

Unless otherwise indicated, the terms “at least” or “about” preceding a series of elements is to be understood to refer to every element in the series. The term “about” depends on the context in which it is used. When preceding a numerical value, it generally includes ±10% of the recited value. For example, in one context, a concentration of about 1 mg/mL includes 0.9 mg/mL to 1.1 mg/mL. Likewise, a concentration range of about 1% to 10% (w/v) can include 0.9% (w/v) to 11% (w/v).

The terms “polypeptide,” “peptide.” and “protein” are used interchangeably to refer to polymers of amino acids of any length, and their salts. The polymer can be linear or branched, can comprise modified amino acids, and can be interrupted by non-amino acids. Except where indicated otherwise, e.g., for the abbreviations for the uncommon or unnatural amino acids set forth herein, the three-letter and one-letter abbreviations, as used in the art, are used herein to represent amino acid residues. Except when preceded with a “D” or in lower case, the amino acid is an L-amino acid. Groups or strings of amino acid abbreviations are used to represent peptides. Except where specifically indicated, peptides are indicated with the N-terminus of the left and the sequence is written from the N-terminus to the C-terminus.

A “retro inverso” peptide has a reversed amino acid sequence, relative to a reference L-amino acid sequence, and is made up of all D-amino acids (inverting the α-center chirality of the amino acid subunits) to help maintain side-chain topology similar to that of the original L-amino acid peptide.

An “isolated” molecule is one that is in a form not found in nature, including those which have been purified.

“Binding affinity” generally refers to the strength of the sum total of non-covalent interactions between a single binding site of a molecule and its binding partner (e.g., a receptor and its ligand, an antibody and its antigen, two monomers that form a dimer, etc.). Unless indicated otherwise, as used herein, “binding affinity” refers to intrinsic binding affinity which reflects a 1:1 interaction between members of a binding pair. The affinity of a molecule X for its partner Y can generally be represented by the dissociation constant (K_{D}). Affinity can be measured by common methods known in the art, including those described herein. Low-affinity binding partners generally bind slowly and tend to dissociate readily, whereas high-affinity binding partners generally bind faster and tend to remain bound longer.

The affinity or avidity of a molecule for its binding partner can be determined experimentally using any suitable method known in the art, e.g., flow cytometry, enzyme-linked immunosorbent assay (ELISA), or radioimmunoassay (RIA), or kinetics (e.g. KINEXA® or BIACORE™ or OCTET® analysis). Direct binding assays as well as competitive binding assay formats can be readily employed. (See, e.g., Berzofsky et al., “Antibody-Antigen Interactions,” in Fundamental Immunology, Paul, W. E., ed., Raven Press: New York, N.Y. (1984); Kuby, Immunology, W. H. Freeman and Company: New York. N.Y. (1992)). The measured affinity of a particular binding pair interaction can vary if measured under different conditions (e.g., salt concentration, pH, temperature). Thus, measurements of affinity and other binding parameters (e.g., K_{D} or Rd, Kₒₙ, K_{off}) are made with standardized solutions of binding partners and a standardized buffer, as known in the art.

An “active agent” is an ingredient that is intended to furnish biological activity. The active agent can be in association with one or more other ingredients. An active agent that is a peptide can also be referred to as an “active peptide.”

An “effective amount” of an active agent is an amount sufficient to carry out a specifically stated purpose.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g., acetate, phosphate, or citrate buffer), a surfactant (e.g., polysorbate), a stabilizing agent (e.g., polyol or amino acid), a preservative (e.g., sodium benzoate), and/or other conventional solubilizing or dispersing agents.

A “subject” or “individual” or “animal” or “patient” or “mammal,” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain embodiments, a subject is successfully “treated” for a disease or disorder if the patient shows total, partial, or transient alleviation or elimination of at least one symptom or measurable physical parameter associated with the disease or disorder.

A “control patient” is a subject that has not received a treatment of the invention. A “control population” or a “population of control patients” is a group of subjects that have not received a treatment of the invention. A control patient or subject in the control population has the same disease or disorder as the subject being compared to the control patient or control population. For example, a clinical outcome of a cancer patient receiving a pharmaceutical composition or method of the invention is compared with the average (median) outcome of subjects having the same type and/or stage of cancer, who did not receive a pharmaceutical composition or method of the invention. In some embodiments, the control patient or patients in the control population have received a treatment other than a treatment of the invention, for example, a standard-of-care treatment.

An “antagonist” is a substance that prevents, blocks, inhibits, neutralizes, or reduces a biological activity or effect of another molecule, such as a receptor or ligand.

The terms “inhibit,” “block,” and “suppress” are used interchangeably and refer to any statistically significant decrease in occurrence or activity, including full blocking of the occurrence or activity. For example, “inhibition” can refer to a decrease of about 10%, 20%, 30%, 40%, 50%, 60%, 70%, 80%, 90% or 100% in activity or occurrence. An “inhibitor” is a molecule, factor, or substance that produces a statistically significant decrease in the occurrence or activity of a process, pathway, or molecule.

A “neoplastic cell” or “neoplasm” typically has undergone some form of mutation/transformation, resulting in abnormal growth as compared to normal cells or tissue of the same type. Neoplasms include morphological irregularities, as well as pathologic proliferation. Neoplastic cells can be benign or malignant. Malignant neoplasms. i.e., cancers, are distinguished from benign in that they demonstrate loss of differentiation and orientation of cells, and have the properties of invasion and metastasis.

A “tumor” or “solid tumor” is a mass of neoplastic cells, such as cancer cells. The terms “advanced,” “metastatic,” and “advanced/metastatic” are used interchangeably to describe a cancer in which malignant cells have migrated from the original tumor to another location, for example, another organ, in a patient's body.

“Pharmacokinetics” or “PK” refers to the study of how an administered substance is processed by the body of a subject. PK determinations include how the substance enters the blood circulation (absorption), is dispersed or disseminated throughout the fluids and tissues of the body (distribution), is recognized and transformed by the body (metabolism), and is removed from the body (excretion). The substance may be a drug, for example, ST316. Pharmacokinetics may be evaluated using various metrics, many of which are calculated based on the quantity of the substance in the body (e.g., in the plasma) at various time points following the administration of the substance.

Time after administration is measured from T₀, which is the time that administration of a single dose of the substance is started. In instances where administration of the pharmaceutical composition is paused and resumed one or more times during the total infusion period, To is the beginning of the total infusion period.

The “total infusion duration” or “total infusion period” is the time from the start of a single dose of the pharmaceutical composition to the end of administration, and includes both periods of infusion and periods of interruption.

“Cₘₐₓ” is a pharmacokinetic metric that refers to the peak plasma concentration of the substance after administration.

“Tₘₐₓ” is a pharmacokinetic metric that refers to the time after the start of administration of the substance (T₀) to reach Cₘₐₓ.

“Tₗₐₛₜ” is a pharmacokinetic metric that refers to the time of last quantifiable concentration of the substance.

“AUC” or “area-under-the-curve” is a pharmacokinetic metric that describes the variation of the concentration of the substance in blood plasma as a function of time. AUC may be calculated for different periods of time, for example, from time zero to specified time t (AUCₜ or AUC₀₋ₜ), from time zero to infinity (AUC_{∞} or AUC_{0-∞}), etc.

“Elimination half-life” or “half-life” or “t_{1/2}” is a pharmacokinetic metric that refers to the time required for the concentration of the substance to reach half of its original value.

“Clearance” is a pharmacokinetic metric that refers to the volume of plasma cleared of the substance per unit time.

“V_{z}” is a pharmacokinetic metric that refers to the volume of distribution during terminal phase.

### II. Peptides and Compositions

### β-Catenin and Antagonist Peptides

The Wnt/β-catenin pathway is a validated target for cancer therapies. Constitutive activation of the Wnt/β-catenin signaling pathway is implicated in carcinogenesis, tumor progression, and poor prognosis of many malignancies (Shang 2017). The complex formed by the interaction between β-catenin and its co-activator, BCL9, drives oncogene expression in multiple cancers due to aberrent Wnt pathway signaling. Disruption of the β-catenin/BCL9 complex has been shown to suppress oncogenic Wnt/β-catenin transcription without impacting β-catenin homeostatic functions (Takada 2012).

The interaction between β-catenin and BCL9 has previously been considered an “undruggable” target due to the inability of small molecules to inhibit complex formation and the inability of antibodies to gain access to the cytoplasm or nucleus to disrupt the interaction (Takada 2012). Peptide antagonism of β-catenin is distinct from these other approaches because it can specifically target the interaction between β-catenin and BCL9 and is selectively toxic to tumor cells where these proteins are hyperactive, amplified, and/or mutated.

In some embodiments, methods of the invention comprise treating a patient having a solid tumor with an effective amount of a peptide antagonist of β-catenin. Preferably, the peptide antagonist of β-catenin inhibits interaction of β-catenin with BCL9. Peptide antagonists of β-catenin can be rationally designed, for example, based on the native sequence of BCL9 with modifications to enhance electrostatic interactions between the peptide antagonist and β-catenin. In particular, peptide antagonists of the interaction between β-catenin and BCL9 can be derived from the homology domain 2 (HD2) of BCL9 and designed to interact with the first armadillo repeat (ARM-1) domain of β-catenin, which is a site utilized by BCL9 but not other β-catenin binding partners. WO 2021/007158 provides numerous examples of such peptides.

The ability of a peptide based on wild-type BCL9 to antagonize the activity of β-catenin, such as to inhibit the interaction of β-catenin with BCL9, can be measured by methods described herein, for instance, in Example 1. The cytotoxic activity of a peptide antagonist of β-catenin can be measured in vitro by known assays and/or in vivo using known tumor models; for example, WO 2019/136125 and WO 2021/007158 describe such assays and models.

The peptide antagonist of β-catenin can be a cell-penetrating peptide. In one embodiment, the peptide comprises a cell-penetrating domain. Numerous cell-penetrating peptide sequences are described and characterized in the literature (see WO 2019/136125). In one embodiment, the peptide is a cyclic peptide. Cyclized peptides, for example, using hydrocarbon staples (Bernal 2007; Bird 2017) or other cyclization methods known in the art, can enter cells via passive diffusion, endocytosis/endosomal escape, or other mechanisms (Dougherty 2019). Peptides can also be delivered to cells via mechanisms that exploit cellular receptors, for example, integrin-targeting, RGD-like sequences. Alternatively, peptides can be encapsulated and delivered to cells in vesicles, such as exosomes or liposomes, or in micelles.

The peptide antagonist of β-catenin can have a modified N-terminus and/or a modified C-terminus. For example, peptide antagonists can optionally include an N-terminal acetyl group and/or a C-terminal amide group. Other examples of optional N-terminal and/or C-terminal groups include hydrophobic groups, such as a linear or cyclic C₂-C₁₈ aliphatic or aromatic hydrocarbon, a naphthyl group, a phenyl group, an octanoyl group, and a valeryl group, including an isovaleryl group. In some embodiments, the peptide antagonist comprises a linker or spacer between the peptide and the hydrophobic group. Such linkers or spacers include, for example, aminohexanoic acid, beta-alanine, substituted alkyls, substituted cycloalkyls, and polyethylene glycol.

ST316 is a novel 25-amino acid peptide antagonist of the interaction between β-catenin and BCL9. ST316 is composed entirely of D-amino acids and is highly stable in the presence of proteolytic enzymes. Due to its resistance to protease degradation. ST316 has a long plasma half-life, and should not be processed and presented by antigen presenting cells, thereby avoiding stimulation of an anti-drug antibody (ADA) response.

ST316 is composed of two domains: (i) a 15 amino acid N-terminal active domain derived from the homology domain 2 (HD2) of BCL9 and designed to interact with the first armadillo repeat (ARM-1) domain of β-catenin, which is a site utilized by BCL9 but not other β-catenin binding partners; and (ii) a 10 amino acid C-terminal domain, which increases solubility and cell penetration. The D-amino acid sequence of ST316 is: FRWLLRQLARLAQLAKLTPVKLTPV (SEQ ID NO: 1). The N-terminal active domain is bolded and the C-terminal cell-penetrating domain is italicized. ST316 also comprises an N-terminal octanoyl group.

ST316 represents the first well-tolerated approach to drugging the Wnt pathway by specifically binding the n-terminus of β-catenin, where it interacts with BCL9, a cofactor that is not required for normal, physiologic function, but is required for the pathologic, oncogenic and immunosuppressive functions of β-catenin.

### Compositions and Administration

In certain aspects, the invention provides a composition, e.g., a pharmaceutical composition, comprising a peptide antagonist of β-catenin, such as ST316. The pharmaceutical composition is suitable for parenteral administration. In one embodiment, ST316 can be in a salt form. Preferably, the composition comprises one or more carriers, diluents, excipients, or other additives. For example, the composition can comprise one or more bulking agents, one or more buffers, one or more pH adjusting agents, and/or one or more diluents.

Aspects of the invention relate to methods of administering a peptide antagonist of β-catenin to a subject. The peptide antagonist of β-catenin is administered parenterally. Parenteral routes of administration include intravenous (IV), intramuscular, intraperitoneal, intrathecal, and subcutaneous. In a preferred embodiment, a pharmaceutical composition comprising ST316 is administered via IV infusion. The pharmaceutical composition can be provided, for example, in an IV fluid comprising normal saline (0.9%), half normal saline (0.45%), or 5% dextrose in water (D5W). In one embodiment, the peptide antagonist of β-catenin is a sterile, lyophilized solid presented in vials for reconstitution, for example, with Sterile Water for Injection USP. The reconstituted peptide can subsequently be diluted for IV administration.

The peptide antagonist of β-catenin can be dosed based on the patient's weight. The peptide antagonist of β-catenin, such as ST316, can be administered to a patient at a dose of about 0.25 mg/kg to about 12 mg/kg. In certain embodiments. ST316 is administered at a dose of about 0.25 mg/kg, about 0.5 mg/kg, about 0.75 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 9 mg/kg, about 10 mg/kg, about 11 mg/kg, or about 12 mg/kg. These amounts can also serve as endpoints for a range of doses to be administered, for example, about 0.75 mg/kg to about 7 mg/kg, about 2 mg/kg to about 4 mg/kg, etc.

Alternatively, the peptide antagonist of β-catenin can be administered at a fixed dose. The peptide antagonist of D-catenin, such as ST316, can be administered to a patient at a dose of about 500 mg to about 1500 mg. In certain embodiments, ST316 is administered at a dose of about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, or about 1500 mg. These amounts can also serve as endpoints for a range of doses to be administered, for example, about 600 ng to about 1100 mg, about 750 mg to about 900 mg, etc.

In embodiments of the invention, the pharmaceutical composition is administered to the subject by intravenous infusion, in which the total infusion duration is no more than about 360 minutes. In some embodiments, the total infusion duration is about 30 minutes to about 240 minutes. For example, the total infusion duration can be 30 minutes, 60 minutes, 90 minutes, 120 minutes, 150 minutes, or 180 minutes, or an intermediate duration, such as 45 minutes, 100 minutes, etc. In certain embodiments, the total infusion duration is about 60 minutes to about 90 minutes, or about 60 minutes to about 120 minutes, or about 90 minutes to about 120 minutes, or about 60 minutes to about 180 minutes.

In some embodiments, infusion of the pharmaceutical composition may be interrupted, i.e., may be temporarily stopped and then resumed. The duration of the interruption may vary, for example, it may be about 15 minutes or less, or about 30 minutes or less, or about one hour or less, or about two hours or less, or about three hours or less, or about four hours or less.

In some embodiments, the pharmaceutical composition may be administered with one or more secondary agents intended to block histamine release, to prevent or ameliorate infusion-related reactions (IRRs), to reduce fever or inflammation, and/or to relieve itching and/or urticaria. IRRs can include, for example, erythema, fevers, chills/rigors, tachycardia, tachypnea, hypotension, and bronchospasm, The one or more secondary agents may be administered concurrently with, before, and/or after the administration of the pharmaceutical composition. The one or more secondary agents may be administered in a separate composition than the pharmaceutical composition, or may be combined with the pharmaceutical composition. In addition, the one or more secondary agents may be administered by the same route as the pharmaceutical composition, or may be administered by a different route (e.g., orally).

Examples of one or more secondary agents for administration with the pharmaceutical composition include, but are not limited to, antihistamines, including H1 antagonists, H2 antagonists, and mast cell degranulation inhibitors (e.g., acrivastine, astemizole, azatadine, azelastine, bepotastine, bromopheniramine, burfroline, cetirizine, chlorzoxazone, chlorpheniramine, cromolyn, cyproheptadine, desloratadine, dexbromphenir amine, diphenhydramine, doxantrozole, epinastine, etodroxizine, famotidine, fexofenadine, forskolin, hydroxyzine, isoproterenol, ketotifen, levocetirizine, loratadine, lodoxamide, mequitazine, methdilazine, mizolastine, nedocromil, olopatadine, oxatomide, pemirolast, pimecrolimus, pirbuterol, pizotifen, proxicromil, ranitidine, terfenadine, terbutaline); leukotriene inhibitors (e.g., montelukast, zafirlukast, zileuton); nonsteroidal anti-inflammatory drugs (NSAIDs) (e.g., ibuprofen, naproxen, aspirin); acetaminophen/paracetamol; corticosteroids (e.g., hydrocortisone, dexamethasone, prednisone, prednisolone); antinausea medications (e.g., prochlorperazine, ondansetron); and saline and/or electrolytes. In one preferred embodiment, the secondary agent is an antihistamine, such as chlorpheniramine or diphenhydramine. In particular embodiments, the secondary agent is selected from the group consisting of acetaminophen/paracetamol, an H1 antagonist, and H2 antagonist, montelukast, an antiemetic, and combinations thereof.

In certain embodiments, the secondary agent is administered during administration of the pharmaceutical composition, such as during the infusion period. In certain embodiments, the secondary agent, such as an antihistamine, is administered to the subject prior to administration of the pharmaceutical composition, such as within about 7 days prior, or within about 6 days prior, or within about 5 days prior, or within about 4 days prior, or within about 72 hour prior, or about 48 hours prior, or about 24 hours prior, or about 8-12 hours prior, or about 6-8 hours prior, or about 4-6 hours prior, or about 2-4 hours prior, or about 1-2 hours prior, or within about 1 hour prior, or immediately prior. In some embodiments, the secondary agent is administered within 24 hours after administration of the pharmaceutical composition. For example, the secondary agent can be administered immediately after, about 0.5-1 hour after, about 1-2 hours after, about 2-4 hours after, about 4-6 hours after, about 6-8 hours after, about 8-12 hours after, or about 24 hours after the completion of administration of the pharmaceutical composition.

The secondary agent can be administered multiple times before, during, and/or after administration of the pharmaceutical composition. Combinations of secondary agents can be administered concurrently or at different times. For example, an antihistamine could be administered before administration of the pharmaceutical composition, and a corticosteroid could be administered after administration of the pharmaceutical composition.

The peptide antagonist is typically administered to the patient multiple times. The peptide antagonist of β-catenin can be administered once weekly, once every two weeks, once every three weeks, once every four weeks, or combinations of these intervals. Total duration of treatment can be at least one, two, three, four, five, six, seven, eight, nine, ten, eleven, or twelve weeks, or at least three, four, five, six, seven, eight, nine, ten, eleven, or twelve months.

In one embodiment, administration of the peptide antagonist of β-catenin can occur once weekly for a duration of at least one week, two weeks, or three weeks (i.e., one, two, or three administrations), six weeks (i.e., six administrations), nine weeks, twelve weeks, three months, six months, nine months, or twelve months. In another embodiment, administration can occur once every two weeks for a duration of at least four weeks (i.e., two administrations), eight weeks (i.e., four administrations), twelve weeks, three months, six months, nine months, or twelve months. In some embodiments, a patient can be administered the peptide antagonist of β-catenin once weekly for a duration of at least one week, two weeks, three weeks, six weeks, nine weeks, twelve weeks, three months, six months, nine months, or twelve months, followed by administration once every two weeks for at least four weeks, eight weeks, twelve weeks, three months, six months, nine months, or twelve months.

Neoadjuvant therapy can be administered to reduce the size or extent of a tumor, or to make a subsequent treatment easier, more effective, or less extensive. In a particular embodiment, the peptide antagonist of β-catenin can be administered as a neoadjuvant prior to, for example, surgery and/or radiation, as a single agent or in combination with a secondary agent.

Pharmacokinetics (PK) and pharmacodynamics of the antagonist of β-catenin can be assessed by standard methods and as described in the Examples. In some embodiments, the antagonist of β-catenin is ST316.

### III. Methods of Treatment

Subjects in need of treatment by the methods of the invention are patients diagnosed with a solid tumor. For example, the subject can have an advanced unresectable and metastatic solid tumor. In one embodiment, the tumor harbors one or more abnormalities in the Wnt/β-catenin signaling pathway. By way of non-limiting example, the one or more abnormalities in the Wnt/β-catenin signaling pathway can comprise one or more mutations in one or more of adenomatous polyposis coli (APC), APC membrane recruitment protein 1 (AMER1). Axin2, β-catenin, RING finger protein 43 (RNF43), and/or T cell factor 7 (TCF7).

In some embodiments, the subject has a locally advanced or metastatic: breast cancer (BC), colorectal cancer (CRC), cholangiocarcinoma (CCA), endometrial cancer, hepatocellular carcinoma (HCC), melanoma, non-small cell lung cancer (NSCLC), ovarian cancer (OC), pancreatic adenocarcinoma (PDAC), or synovial sarcoma (SS). In certain embodiments, the disease is refractory or intolerant to other available therapies that would impact survival. In particular embodiments of the invention, administration of ST316 can inhibit tumor growth, reduce tumor volume, or a combination thereof.

In one embodiment, the patient has been diagnosed with BC. More than 50% of BC tumors have been shown to harbor abnormalities of Wnt signaling (Lin 2000). Triple-negative breast cancer (TNBC) is a particularly aggressive form of BC that generally displays poorer prognosis compared to other BC subtypes and accounts for 10-15% of all BC cases (Won 2020). TNBC patients do not respond to hormonal and targeted therapies due to the lack of hormone receptors and human epidermal growth factor receptor 2 (HER2). Chemotherapy remains the standard of care, despite its limited benefit. Thus, the medical need for more effective agents in this clinical setting remains very high. In TNBC, Wnt pathway mutations are known to drive tumorigenesis and metastasis, lead to poor clinical outcomes and increase the likelihood of developing lung and brain metastases (Pohl 2017).

In one embodiment, the patient has been diagnosed with CRC. CRC is one of the most diagnosed cancers in the world, ranking second in women and third in men globally (Sung 2021). Approximately 95% of metastatic CRC (mCRC) patients have microsatellite stable disease (MSS), and are ineligible for treatment with immune checkpoint inhibitors. This patient population relies on modestly efficacious chemotherapy and targeted therapy regimens, making it an area of significant unmet medical need. More than 90% of CRC tumors have been shown to harbor abnormalities of Wnt signaling. These have been demonstrated to drive disease initiation, progression and metastasis (Nie 2020).

In one embodiment, the patient has been diagnosed with CCA. Also known as biliary tract cancer, CCA refers to a heterogeneous group of gastrointestinal cancers that arise from the bile ducts, the gallbladder, or the ampulla of Vater. These cancers are often difficult to diagnose, due to their anatomical location and the paucity or non-specificity of their symptoms; therefore, >75% of CCA patients present with advanced, unresectable disease (Lamarca 2014, Takahashi 2013). Advanced unresectable CCA represents an area of unmet medical need, due to its very aggressive nature, limited treatment options, and poor prognosis, particularly after first line treatment, generally with a combination of gemcitabine and cisplatin (Valle 2010). Clinical and preclinical studies have suggested the key role of activation of the Wnt/β-catenin signaling pathway in induction and progression of CCA (Zhang 2020).

In one embodiment, the patient has been diagnosed with OC. OC is the fifth leading cause of cancer death in women, and the leading cause of death among gynecologic cancers (ACS 2022). Two thirds of patients with OC are diagnosed at stages 3 and 4, accounting for the relatively low 5-year survival rate of 39% and 17% for stage 3 and 4 tumors, respectively (NCCN 2022). Advanced OC represents an area of unmet medical need due to its very aggressive nature, limited treatment options, and poor prognosis. Wnt activity has been shown to correlate with histological grade, epithelial to mesenchymal transition, chemotherapy resistance, and poor prognosis in OC (Teeuwssen 2019).

In one embodiment, the patient has been diagnosed with endometrial cancer. Endometrial cancer is the most common gynecological cancer in the industrialized world and is increasing in women of all ages, at least partly due to increased incidence of obesity and resulting hyperinsulinemia (Moore 2017; Parrish 2022)). Older women experience a higher risk of recurrence and higher mortality rates (Moore 2017). Mutations in β-catenin are associated with worse outcomes in endometrial cancer patients, including higher incidence of recurrence and lower survival rates (Parrish 2022).

Efficacy of treatment can be evaluated by one or more known measures. For example, patients subjected to methods of the invention can experience outcomes including extended survival, improved progression-free survival, improved duration of response, longer remission, reduced risk of relapse, and/or improved tumor response to treatment with a peptide antagonist of β-catenin, compared with the same outcome(s) in patients not subjected to methods of the invention, i.e., control patients. An outcome in a patient treated by a method of the invention can be compared, for example, to the median outcome in a population of control patients. The population of control patients can be administered, for example, a regimen selected from the group consisting of a placebo, surgery, radiation, chemotherapy, immunotherapy, hormone-based therapy, targeted therapy, and combinations thereof. Comparisons can be analyzed statistically using, for example, the Wilcoxon signed rank test or the Kaplan-Meier method.

In one embodiment, outcome in a patient receiving a peptide antagonist of 3-catenin, such as ST316, optionally in combination with a standard-of-care treatment, is compared with median outcome in control patients receiving a placebo. In one embodiment, outcome in a patient receiving a peptide antagonist of β-catenin, such as ST316, is compared with median outcome in control patients receiving a standard-of-care treatment.

Tumor response to treatment can be assessed, for example, by measuring tumor burden and/or tumor regression. Response to treatment compares one or more measures of efficacy after a treatment regimen, as compared to baseline, e.g., prior to treatment with ST316. A baseline assessment is preferably performed within 24, 48, or 72 hours, or within 1, 2, 3, or 4 weeks prior to the first treatment with a peptide antagonist of β-catenin. In a one preferred embodiment, a baseline assessment is performed within one week prior to the first ST316 treatment.

“Tumor burden” is the total mass or total size of cancerous tissue in a patient's body. Tumor response can be evaluated by measures including objective response rate, including partial response and/or complete response, stable disease, disease control rate, duration of disease control, and duration of response. These parameters can be determined, for example, by revised Response Evaluation Criteria in Solid Tumors (RECIST 1.1) (Eisenhauer 2009).

Objective response rate assesses reduction of tumor size, for example, tumor diameter, which can be determined by clinical examination and/or imaging. Where a patient has multiple tumors, tumor size can optionally be expressed as the average diameter of all tumors or by the sum of diameters of all tumors. Superficial tumors can be measured clinically, for instance, using calipers or by photography and ruler measurement. Imaging methods include computed tomography (CT), typically with contrast; X-ray; magnetic resonance imaging (MRI); and positron emission tomography (PET), such as (18)F-fluorodeoxyglucose PET. In one preferred embodiment, CT is utilized to assess tumor response, for example, in a BC patient or a melanoma patient. Accordingly, in one aspect, the invention provides a method of reducing tumor burden, i.e., tumor mass and/or tumor size, in a patient, the method comprising administering to the patient a peptide antagonist of β-catenin, such as ST316. Reduction in tumor burden is measured relative to baseline.

In certain embodiments, particularly those in which assessment is by RECIST 1.1, disease control rate defines the level of tumor response as the best of the following: complete response (CR), which is the disappearance of tumor(s); partial response (PR), which is a decrease of at least 30%, in the size of tumor(s); stable disease (SD), in which the change in tumor size is decreased by less than 30% or increased by less than 20%; or disease progression, which is an increase of at least 20%, in tumor size and/or new lesions. Patients treated by methods of the invention can experience CR, PR, or SD.

Duration of disease control is the length of time from achievement of a response (CR or PR) or SD until disease progression. Duration of response is the length of time from the achievement of a response until disease progression. i.e., the period in which a tumor does not grow or spread, or death. Duration of response in patients receiving ST316 treatment can be, for example, at least 4, 6, 8, 10, or 12 weeks, at least 4, 6, 8, 10, 12, 16, 18, or 24 months, or at least 3, 4, or 5 years. Patients treated by methods of the invention can experience increased duration of disease control or increased duration of response. Accordingly, in one aspect, the invention provides a method of increasing the duration of disease control or duration of response in a patient, the method comprising administering to the patient a peptide antagonist of D-catenin, such as ST316. Increase in duration of disease control or duration of response is measured relative to the median duration of disease control or duration of response, respectively, in a control population.

Survival can be assessed as overall survival. i.e., the length of time a patient lives, as progression-free survival, i.e., the length of time a patient is treated without progression or worsening of the disease, or as event-free survival, i.e., the length of time that a patient remains free of complications or negative events such as relapse or disease progression. Survival is from the date that treatment commences. Overall survival, median overall survival, progression-free survival, median progression-free survival, event-free survival, and median event-free survival can be calculated, for example, by Kaplan-Meier analysis, based on the response to treatment.

Accordingly, in one aspect, the invention provides a method of increasing overall survival in a patient, the method comprising administering to the patient a peptide antagonist of β-catenin, such as ST316. Increase in overall survival can be measured relative to the median overall survival in a control population. Alternatively, an increase compared to a control population in the percentage of patients who survive for a given time period (e.g., 6 months or 12 months) indicates an increase in overall survival.

In another aspect, the invention provides a method of increasing progression-free survival in a patient, the method comprising administering to the patient a peptide antagonist of β-catenin, such as ST316. Increase in progression-free survival can be measured relative to the median progression-free survival in a control population. Alternatively, an increase compared to a control population in the percentage of patients who are relapse-free for a given time period (e.g., 6 months or 12 months) indicates an increase in progression-free survival.

In a further aspect, the invention provides a method of increasing event-free survival in a patient, the method comprising administering to the patient a peptide antagonist of β-catenin, such as ST316. Increase in event-free survival is measured relative to the median event-free survival in a control population. Alternatively, an increase compared to a control population in the percentage of patients who are event-free for a given time period (e.g., 6 months or 12 months) indicates an increase in event-free survival.

A patient is successfully treated according to the methods of the invention if the patient experiences or displays at least one of the following outcomes after administration of a peptide antagonist of 3-316, such as ST316:
undetectability of the tumor (or at least one tumor, if multiple tumors are present at baseline);
at least about 10%, 15%, 20%, 25%, 30%, 35%, 40%, 45%, 50%, 55%, 60%, 65%, 70%, 75%, 80%, 85%, 90%, or 95% reduction in tumor size compared to baseline;
no significant increase (e.g., less than 20% or less than 30%) in tumor size compared to baseline;
significantly increased duration of response, optionally compared with median duration of response of a population of control patients:
significantly increased duration of disease control, optionally compared with median duration of disease control of a population of control patients;
significantly increased progression-free survival, optionally compared with median progression-free survival of a population of control patients;
significantly increased overall survival, optionally compared with median overall survival of a population of control patients.

### IV. Methods of Preparation

Peptide antagonists of β-catenin can be chemically synthesized, for example, using solid-phase peptide synthesis or solution-phase peptide synthesis, or a combination of both. Synthesis may optionally occur as fragments of the peptide that are subsequently combined either chemically or enzymatically.

Alternatively, peptide antagonists of β-catenin can be expressed using recombinant methods. For example, nucleic acid molecules encoding ST316 can be constructed by chemical synthesis using an oligonucleotide synthesizer. Nucleic acid molecules can be designed based on the amino acid sequence of ST316 and selection of those codons that are favored in the host cell in which the recombinant ST316 will be produced. Standard methods can be applied to synthesize a nucleic acid molecule encoding a peptide antagonist of β-catenin, such as ST316.

Once prepared, the nucleic acid encoding the peptide can be inserted into an expression vector and operably linked to an expression control sequence appropriate for expression of the peptide in a desired host. In order to obtain high expression levels of the peptide, the nucleic acid can be operably linked to or associated with transcriptional and translational expression control sequences that are functional in the chosen expression host.

A wide variety of expression host/vector combinations can be employed to anyone known in the art. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, wider host range plasmids, such as M13, and filamentous single-stranded DNA phages.

Suitable host cells include prokaryotes, yeast, insect, or higher eukaryotic cells under the control of appropriate promoters. Prokaryotes include gram negative or gram positive organisms, for example E. coli or bacilli. Higher eukaryotic cells can be established or cell lines of mammalian origin, examples of which include Pichia pastoris, 293 cells, COS-7 cells, L cells, C127 cells, 3T3 cells, Chinese hamster ovary (CHO) cells, HeLa cells, and BHK cells. Cell-free translation systems can also be employed.

Peptides can be purified using methods that include, for example, reverse-phase high-performance liquid chromatography (RP-HPLC), multicolumn countercurrent solvent gradient purification (MCSGP), hydrophobic interaction chromatography (HIC) and ion-exchange chromatography.

### Examples

Embodiments of the present disclosure can be further defined by reference to the following non-limiting examples. It will be apparent to those skilled in the art that many modifications, both to materials and methods, can be practiced without departing from the scope of the present disclosure.

### Example 1. ST316 Binds to β-Catenin and Disrupts its Interaction with BCL9

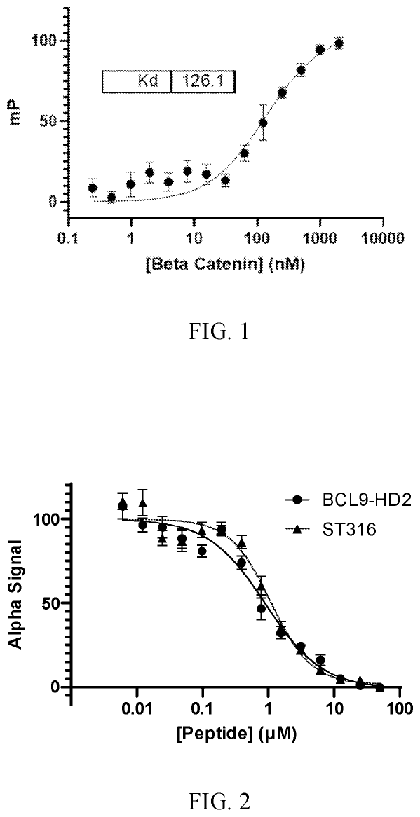

We used a fluorescence polarization assay to measure binding between ST316 conjugated to FITC and recombinant β-catenin. Increasing concentrations of full-length β-catenin (0.24 nM-2000 nM) were incubated with 12.5 nM ST316-FITC for one hour at room temperature. Fluorescence polarization was measured with a SpectraMax® plate reader. ST316 binds to β-catenin Arm1 with a K_{D} of approximately 126.1 nM (FIG. 1). The binding affinity of BCL9 and β-catenin is approximately 200 nM, indicating that ST316 binds β-catenin with equal affinity to or higher affinity than the endogenous ligand.

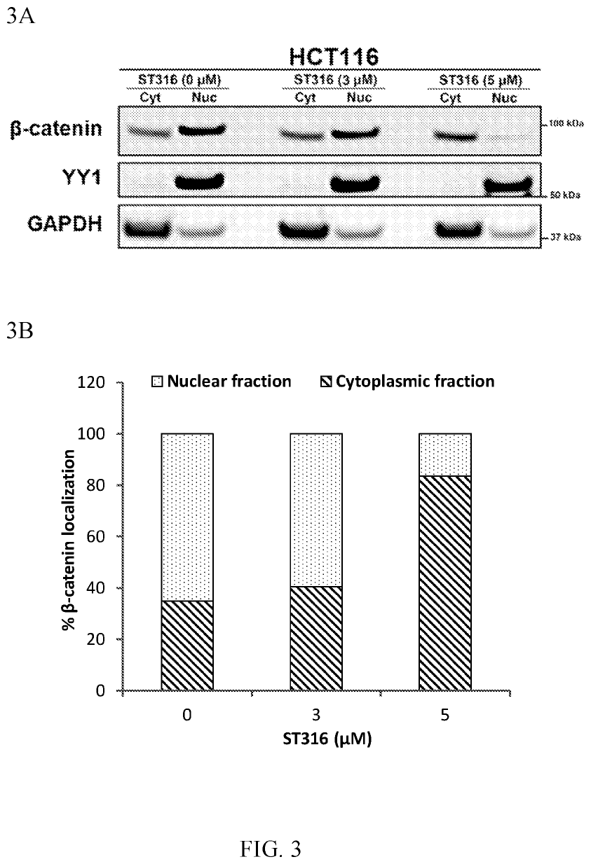

We used an ALPHA assay to quantify the interaction of BCL9 and β-catenin. ALPHA assay was performed in an assay buffer composed of 20 mM MES (pH 6.5), 60 mM NaCl, 0.01% BSA, 1 mM DTT, 0.05% Tween-20. ST316 was serially diluted to a final concentration ranging from 50 M to 6 nM and added to the plate, followed by the addition of 25 nM full-length recombinant β-catenin. The plate was incubated for 1 hour at room temperature. A 26-amino acid, C-terminally biotinylated BCL9 HD-2 peptide was added to each well at final concentration of 50 nM, followed by the addition of a rabbit polyclonal anti-β-catenin antibody (Ab1605) (final concentration 50 ng/mL). The donor and acceptor beads(PerkinElmer AlphaScreen Streptavidin Donor Beads 6760007 and Protein A Acceptor Beads 6760136, respectively) beads were added to the mix at a final concentration of 10 g/mL each, and the plate was protected from light and incubated for one hour at room temperature on a plate rocker. The alpha signal was then measured using a SpectraMax® plate reader. Results showed that both ST316 and HD2 peptide inhibit the interaction of BCL9 and β-catenin with an IC₅₀ of about 1 μM (FIG. 2).

### Example 2. In Vitro Effects of ST316

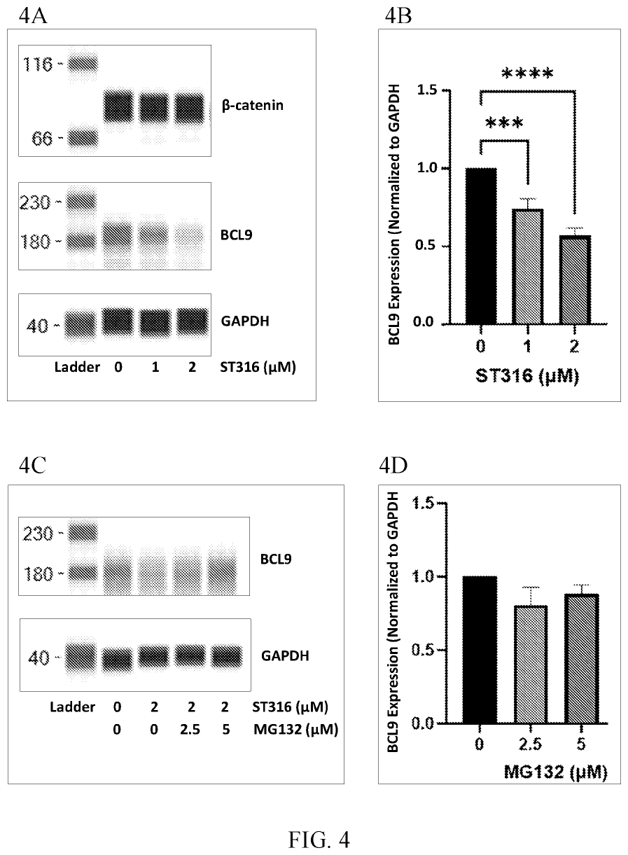

We examined the effect of ST316 on nuclear localization of β-catenin in HCT116 cells. Briefly, cells were cultured in T25 culture flasks with McCoy's 5A media (with 10% FBS and Mycozap), and treated with vehicle only, 3 μM ST316, or 5 μM ST316 for 24 hours. Cells were fractionated by first harvesting followed by incubating in lysis buffer with DTT and a protease inhibitor cocktail. Next, a solution of 10 IGEPAL CA-630 was added to the cells. Cells were spun down to collect the supernatant cytoplasm fraction. The remaining pellet was resuspended in extraction buffer containing DTT and a protease inhibitor cocktail. Cells were spun down and the nuclear supernatant was collected and analyzed by Western blot (FIG. 3A). Treatment with 5 μM ST316 significantly reduced nuclear localization of β-catenin. FIG. 3B indicates the percentage of nuclear and cytoplasmic β-catenin in the Western blot image as quantified by ImageJ analysis software.

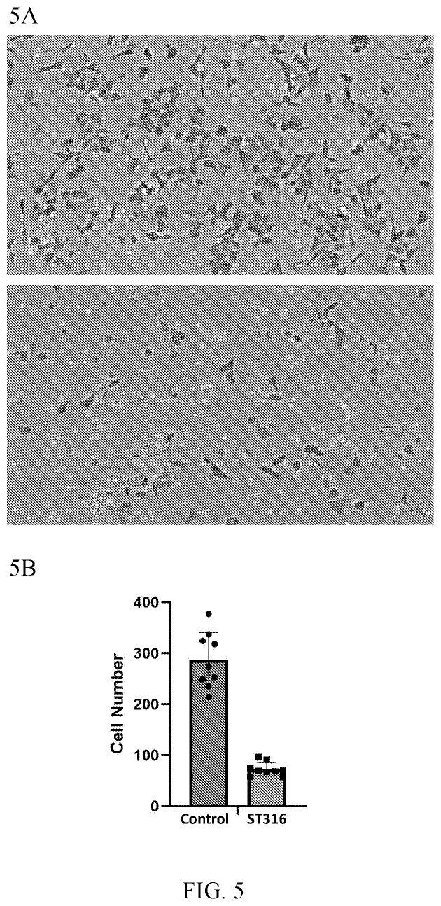

To examine the effect of ST316 on proteasomal degradation of BCL9, Colo320 colorectal cancer cells were incubated with ST316 for 24 hours, with or without the proteasome inhibitor MG132. No impact on cell viability was observed. Protein expression was analyzed using the Jess Protein Detection System. Results are shown in FIG. 4A-4D. Similar results were observed in HCT116 cells.

### Example 3. ST316 Displays Inhibitory Effect on Invasion of Cancer Cells In Vitro

We used an in vitro Boyden chamber assay to investigate the inhibitory effect of ST316 on invasion of neoplastic cells. HCT116 colorectal cancer cells (2×10⁵) were equilibrated in serum-free cell culture medium for two hours in a Matrigel-coated invasion-plate insert, after which complete growth medium plus 10% fetal bovine serum was added to the bottom well of each insert as a chemoattractant. Cells were then exposed to 1 μM ST316 or negative control peptide for 24 hours. No significant impact on cell viability was observed under these conditions.

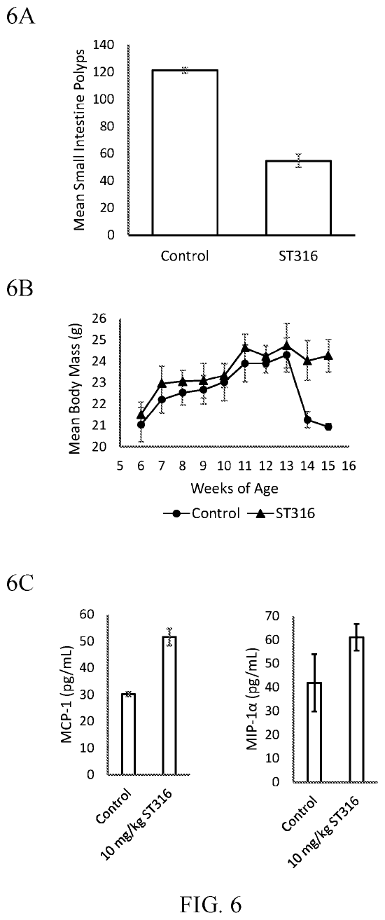

For analysis, the medium in the inserts was aspirated, and the upper layer of the membrane was removed. Following fixation with MeOH and staining with crystal violet, membranes were removed and mounted on a glass slide. Image acquisition of the membranes with invaded cells was made on a Nikon Eclipse T2S microscope at 20× magnification using a Nikon Digital Sight 1000 Microscope. Cell counts were determined manually using ImageJ. Data represents mean and standard error of three biological replicates containing at least three representative fields of view. Results are shown in FIG. 5A-5B.

### Example 4. Administration of ST316 Inhibits Tumor Growth In Vivo

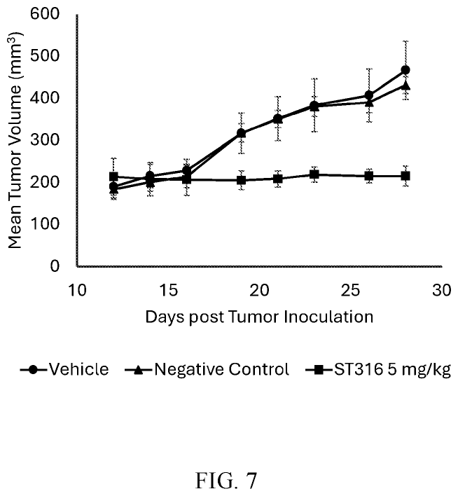

We examined the effect of ST316 on tumor formation in APC^{min/+} mice, which harbor a mutation in APC required for regulation of β-catenin concentration. APC^{min/+} mice are predisposed to cachexia, and develop intestinal adenoma. Six-week-old female APC^{min/+} mice were administered ST316 at a dose of 10 mg/kg once weekly via subcutaneous injection (n=3 per group). Mice received a total of 10 doses. Body weight was measured three times per week for the duration of the study. After week 16, mice were euthanized, plasma was collected for cytokine analysis, and small intestine was collected for adenoma quantification. ST316 treatment resulted in a 55% decrease in adenoma formation (FIG. 6A), prevented the onset of cachexia (FIG. 6B), increased serum pro-inflammatory CCL2/CCL4 cytokine expression (FIG. 6C), compared with mice not receiving ST316 treatment.

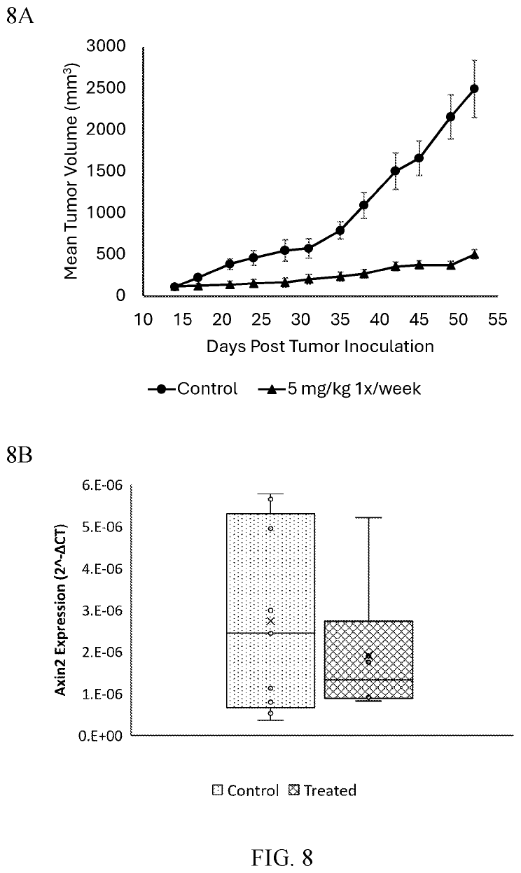

In addition, we examined the effect of ST316 on tumor volume in a subcutaneous tumor model using colorectal cancer cells. Briefly, HCT116 cells (5×10⁵), suspended 1:1 in Matrigel, were implanted via subcutaneous injection into the axilla of female NOD/SCID mice. ST316 was administered at a dose of 5 mg/kg via subcutaneous injection three times weekly for three weeks. Dosing was initiated on day 12 post tumor inoculation, with average starting tumor volume of about 200 mm³. Tumor volume was monitored three times weekly. ST316 treatment resulted in 99% inhibition of tumor growth relative to vehicle or a control peptide (FIG. 7).

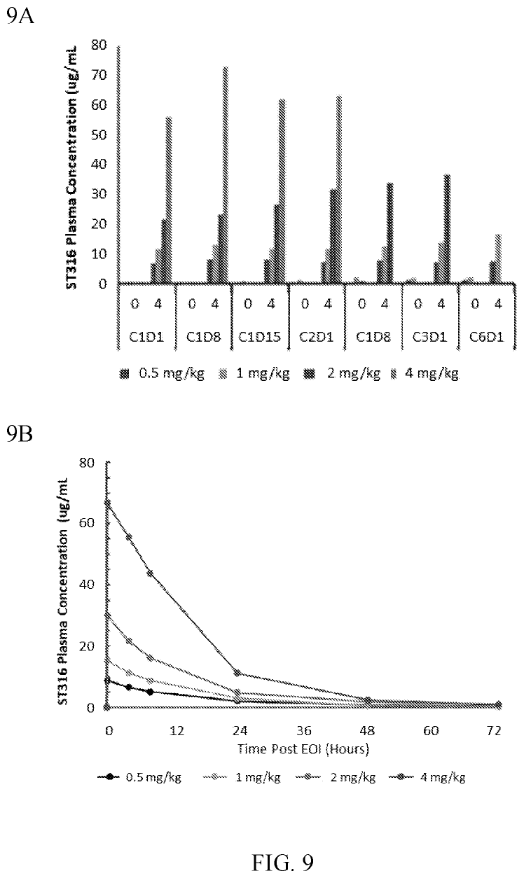

We also examined the effect of ST316 on tumor volume in a subcutaneous tumor model using triple-negative breast cancer cells. Briefly, 4T1-luc cells (5×10⁵), suspended 1:1 in Matrigel, were implanted via subcutaneous injection into the axilla of female NOD/SCID mice. ST316 was administered at a dose of 5 mg/kg via subcutaneous injection once weekly for eight weeks. Dosing was initiated on day 6 post tumor inoculation, with average starting tumor volume of about 100 mm³. Tumor volume was monitored three times weekly. ST316 treatment resulted in 84% inhibition of tumor growth relative to a control peptide (FIG. 8A). Tumors were collected at day 60 and analyzed for Axin2 expression by quantitative PCR (n=3 per group). Results are shown in FIG. 8B.

### Example 5. ST316 is Safe and Well-Tolerated in Animal Models

### Safety Pharmacology

The potential pharmacological effects of ST316 on the cardiovascular (minipigs), respiratory (rats and minipigs) and central nervous systems (CNS) (rats and minipigs) were investigated in 28/29-day GLP toxicology studies. No ST316-related effects were observed on body weight, food consumption, ophthalmologic examinations, respiratory or the CNS in rats or minipigs. Additionally, in rats there were no ST316-related effects on Functional Observational Battery (FOB), and in minipigs there were no ST316-related effects on the electrocardiogram (ECG) rhythm, morphology, or quantitative measurements for QRS, RR, PR, QT, or QTcF intervals.

ADA formation was not detected in a non-GLP toxicology study in C57BL/6 mice following weekly dosing for 10 weeks or in a GLP toxicology study in minipigs following weekly dosing for 4 weeks (5 doses), and was within the 5% error rate of the qualified assay in a GLP toxicology study in rats following weekly dosing for 4 weeks (5 doses). One positive sample in the GLP minipig study (96 total samples) and 2 positive samples (1%) at the 10 mg/kg dose level in the GLP rat study from a control animal that was not exposed to ST316 (198 total samples) were observed.

### Tolerability

Findings in the GLP toxicology studies were mostly limited to the injection site in both rats and minipigs. None of the definitively ST316-related observations in animals receiving ST316 through the catheter were considered adverse. In minipigs, no test article-related pathologic findings were observed.

### Assessment of Pseudoallergic Reactions

The DLT associated with ST316 exposure in MTD studies was a dose-dependent pseudoallergic infusion-related reaction (IRR) observed in mice, rats, and minipigs. Clinical signs of pseudoallergic reaction in mice can include decreased body temperature, reduced activity, and piloerection. The nonclinical toxicology of IV administered ST316 was characterized in a 29-day GLP toxicology study in rats and a 28-day GLP toxicology study in minipigs, which were supported by maximum tolerated dose (MTD) and 7-day repeat dose-range finding (DRF) studies in the same species.

Toxicology studies to characterize the mechanism of the ST316 pseudoallergic IRR demonstrated that the ST316 reaction is mediated by mast cell activation and histamine release, and is mitigated by pretreatment with antihistamines (ranitidine and pyrilamine) and/or the leukotriene receptor antagonist (montelukast).

### Example 6: Administration of ST316 to Patients with Solid Tumors

An open-label, two-part, phase 1-2 study is being conducted to determine the safety, tolerability, pharmacokinetics (PK), pharmacodynamics, and proof-of-concept efficacy of ST316 administered intravenously to subjects with selected advanced solid tumors likely to harbor abnormalities of the Wnt/β-catenin signaling pathway. Wnt/β-catenin pathway abnormalities are an appropriate target because of their impact on carcinogenesis, tumor progression, and prognosis of the tumor types chosen for this study, as well as their impact on immune constituents of the tumor microenvironment. Furthermore, nonclinical studies have demonstrated ST316-specific antagonism of β-catenin and potent in vitro/in vivo activity in breast and colon cancer models. This study utilizes a novel peptide drug with a new mechanism of action in subjects who have exhausted standard-of-care treatments. The study consists of two phases: a phase 1 dose escalation/regimen exploration phase and a phase 2 expansion phase.

### Dose Escalation Phase

The dose escalation phase employs a standard approach of evaluating safety, tolerability, and selection of a recommended phase 2 dose in a broad range of advanced unresectable and metastatic solid tumors, including subjects diagnosed with locally advanced or metastatic BC, CCA, CRC, endometrial cancer, HCC, melanoma, NSCLC, OC, PDAC, or SS, whose disease is refractory or intolerant to all available therapies that would impact survival. Table 1 shows the study design of the dose escalation phase.

TABLE 1
Phase 1 Dose Escalation Overview

| | |
|---|---|
| POPULATION | Relapsed/refractory solid tumor types with known |
| (EST. N = 25) | abnormalities of the Wnt/β-catenin signalling pathway |
| OBJECTIVES | Understand safety, PK, PD |
| | Refine dosing |
| DESIGN | 3 + 3 |
| | Monotherapy |
| | 1x/weekly IV |
| | 12 mg/kg (N = 3-6) |
| | 8 mg/kg (N = 3-4) |
| | 4 mg/kg (N = 4) |
| | 2 mg/kg (N = 3) |
| | 1 mg/kg (N = 4) |
| | 0.5 mg/kg (N = 3) |

ST316 is administered intravenously. The dose escalation cohorts are recruited using a standard 3+3 design. The initial dose infusion duration is 60-180 minutes for all subjects, which can be modified in subsequent doses, based at least in part on the presence or absence of IRRs.

The dose cohorts are 0.5, 1, 2, 4, 8, and 12 mg/kg once weekly (QW), optionally with dose cohorts of 0.25, 0.75, 1.5, 3, and 6 mg/kg and flat dosing. One treatment cycle consists of 21 days QW.

Subjects continue treatment until disease progression, consent withdrawal, or lack of clinical benefit as determined by the treating physician. Patients can continue treatment beyond progression if they are clinically stable. If repeat imaging shows a reduction or stabilization in the tumor burden compared to the initial scan demonstrating PD, treatment is continued as scheduled. If repeat imaging confirms PD, patients are discontinued from therapy. In determining whether or not the tumor burden has increased, stabilized or decreased, Investigators consider all target lesions as well as non-target lesions.

The National Cancer Institute common terminology criteria for adverse events (CTCAE), v5.0 is used for grading toxicities. Safety assessments include adverse events, serious adverse events, physical examinations, vital sign measurements, Eastern Cooperative Oncology Group (ECOG) status, clinical safety laboratory evaluations (hematology, serum chemistry, and hepatic panels, coagulation, and urinalysis) and electrocardiograms. All subjects are followed for safety at least 30 days following the last dose of ST316.

To mitigate potential IRRs, subjects are pretreated before the first dose of ST316 with (i) daily montelukast (10 mg PO) starting at least 2 days before ST316 administration and on the day of administration; and (ii) both an H1 and H2 antagonist on the day of dosing with ST316, at least 30 minutes prior to the start of infusion. If oral antihistamines are used, they are administered earlier based on their predicted Cₘₐₓ. Preferred H1/H2 antagonists include famotidine (20 mg PO or 20-40 mg IV) plus chlorphenamine (10 mg IV/PO) or diphenhydramine (50 mg PO/IV).

Subjects are monitored for IRRs for 24 hours after the first administration pf ST316, for 4 hours after the 2^{nd} infusion, and 2 hours after subsequent infusions. Treatment of IRRs includes, for example, pausing and slowing the infusion rate, and/or administration of antihistamines, such as diphenhydramine, leukotriene receptor antagonists, acetaminophen, nonsteroidal anti-inflammatory drugs (NSAIDs), IV fluids, anti-emetics, oxygen, bronchodilators, and, if other measures are inadequate, corticosteroids. Table 2 provides non-limiting examples of agents for treating IRRs.

TABLE 2
Agents to Treat IRR

| Agent | Dose | | Route |
|---|---|---|---|
| Acetaminophen | 1,000 | mg | IV or PO |
| Diphenhydramine | 25-50 | mg | IV or PO |
| Ranitidine | 50 | mg | IV or PO |
| Ibuprofen | 200-400 | mg | PO |
| Naproxen | 500 | mg | PO |
| Normal saline 0.9% | 1 L or PRN | | IV ( warmed) |
| Prochlorperazine | 5-10 | mg | IV or PO |
| Ondansetron | 4-16 | mg | IV or PO |

Available safety data as well as PK, PD, and efficacy data, are considered to select the maximum tolerated dose and the recommended phase 2 dose, and to potentially model the flat dose cohort(s), if applicable. Once a decision as to the recommended phase 2 dose and regimen is made, the study proceeds to the expansion phase.

### Efficacy

Patients in Cohorts 1-4 are receiving treatment and have undergone a first assessment, as summarized in Table 3. Results show two CRC patients with stable disease, with one patient exceeding 4 months and one patient exceeding 6 months.

TABLE 3
Phase 1 Dose Escalation Summary

| | Dose | Cancer | Enrollment | |
|---|---|---|---|---|
| Cohort | (mg/kg) | Type | Date | Status |
| 1 | 0.5 | NSCLC | Jun. 2023 | Progressed |
| 1 | 0.5 | NSCLC | Jun. 2023 | SD (18 wks); Off Study |
| 1 | 0.5 | CRC | Jul. 2023 | Progressed |
| 2 | 1 | PDAC | Aug. 2023 | Progressed |
| 2 | 1 | TNBC | Sep. 2023 | Progressed |
| 2 | 1 | NSCLC | Sep. 2023 | Progressed |
| 2 | 1 | CRC | Sep. 2023 | SD (27+ wks); On |
| | | | | Study |
| 3 | 2 | CRC | Nov. 2023 | SD (18+ wks); On |
| | | | | Study |
| 3 | 2 | CRC | Nov. 2023 | Progressed |
| 3 | 2 | CRC | Dec. 2023 | Progressed |
| 4 | 4 | PDAC | Jan. 2024 | Progressed |
| 4 | 4 | CRC | Jan. 2024 | On Study |
| 4 | 4 | CRC | Feb. 2024 | On Study |
| 4 | 4 | CRC | Feb. 2024 | On Study |

### Safety

The Wnt pathway, which ultimately signals through β-catenin, has an important role in healthy individuals. β-catenin is an active transcription factor that controls certain stem cell populations and organ regeneration. Given that the Wnt pathway is also critical to many cancers, there have been many clinical attempts at drugging this pathway, despite “on target off tissue” concerns. Previous attempts include: pan-Wnt inhibitors, which suffer on-target toxicity to gastrointestinal stem cells and bone (Kahn 2014; Shaw 2019); inhibition of Wnt pathway members such as Frizzled, Tankyrase. Dickkopf-1 and Porcupine; and disruption of β-catenin transcription complex by targeting T-cell factor/lymphoid enhancer factor. All of these approaches have resulted in toxicity.

Significantly, safety data from Cohorts 1-4 reveals no dose-limiting toxicities, no infusion-related reactions, and no ST316-related severe adverse events.

### Pharmacokinetics and Pharmacodynamics

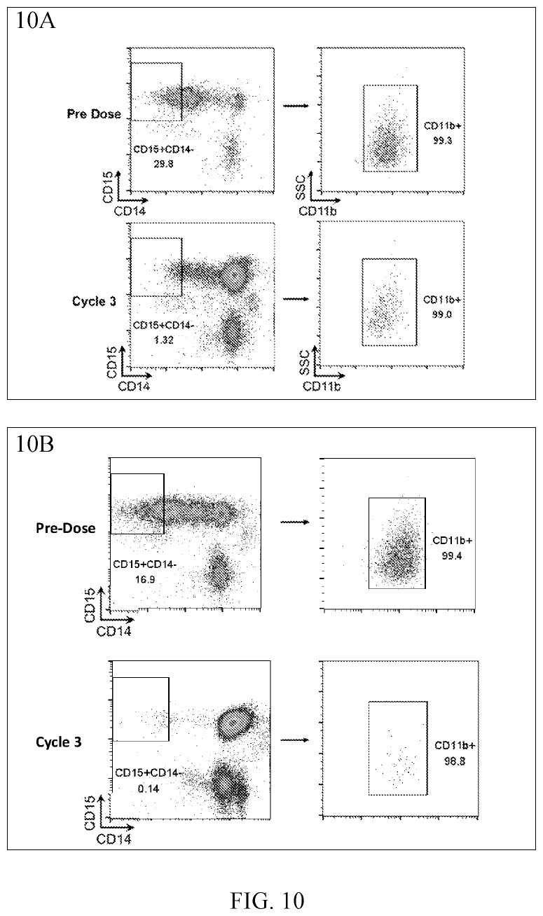

Pharmacokinetics (PK) data from the first four cohorts of patients show relatively linear PK parameters, with no significant accumulation or enhanced clearance over time. The AUC₍₀₋₁₆₈₎ for Cohort 2 exceeded the predicted threshold for biologic activity from modeling. Results are shown in Table 4 and FIG. 9A-9B.

TABLE 4
Pharmacokinetics Summary

| | Infusion | Cₘₐₓ | AUC₍₀₋₁₆₈₎ |
|---|---|---|---|
| Dose (mg/kg) | Duration | (μg/mL) | (hr* μg/mL |
| 0.5 | 1.5 hr. | 8.8 | 159.1 |
| 1 | 1.5 hr. | 15.3 | 237.7 |
| 2 | 1.5 hr. | 30.1 | 461.6 |
| 4 | 1.5 hr. | 66.7 | 1085 |

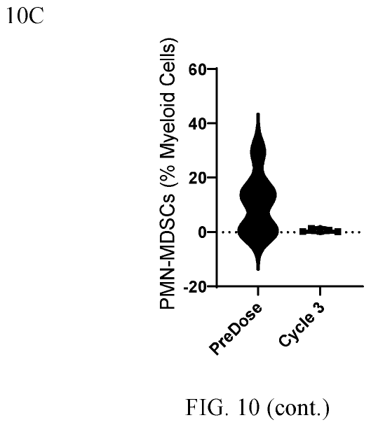

Blood samples taken from patients pre- and post-ST316 exposure analyzed for immune cell populations and show a clear pharmacodynamic effect of ST316 on the WNT/β-catenin pathway. ST316 treatment resulted in reduction of the immune-suppressive polymorphonuclear (PMN) myeloid-derived suppressor cell (MDSC) population (FIG. 10A-10C). PMN-MDSC exhibit a CD3-CD19-CD14-HLADRlowCD11b+CD15+ phenotype.

Taken together, the clinical data demonstrates that the delivered doses of ST316 are pharmacological, and that ST316 is safe, hits the target, and positively affects patients' disease.

### Expansion Phase

The expansion phase consists of three colorectal cancer cohorts. Fifteen subjects will be enrolled in Cohort 1 with ST316 monotherapy. If one or more subjects has a positive efficacy criterion defined as stable disease (SD) more than four months; or any response, partial response (PD) or complete response (CR), then 15 more patients will be recruited to Cohort 1. If Cohort 1 is safe, Cohorts 2 and 3 will be recruited.

Cohort 2 will recruit 15 patients in second line to be treated with ST 316 in combination with bevacizumab and FOLFIRI. If two or more subjects have a response (PR or CR) and/or PFS for more than seven months, additional subjects may be recruited.

Cohort 3 will recruit 15 patients with up to four previous lines of therapy to be treated with ST 316 in combination with fruquintinib. If one or more subjects has a response (PR or CR) and/or PFS for more than four months, additional subjects may be recruited.

Additional tumor types (e.g., endometrial cancer, melanoma, TNBC, ovarian cancer) and combinations may be added in the expansion phase, based on efficacy signals during the dose-escalation and expansion phases.

### Pharmacokinetics Assessment

ST316 PK in plasma are assessed using standard methods. A full plasma PK profile, including AUCₜ, Cₘₐₓ, t_{1/2}, AUC_{∞}, and tₘₐₓ, is obtained and analyzed using non-compartmental methods for all subjects in the dose-escalation phase. Blood samples are drawn pre-dose, at the end of infusion, and at various timepoints after infusion during the first cycle and less frequently thereafter.

### Pharmacodynamics Assessment

Blood and tumor samples are collected for pharmacodynamics assessment, which can include circulating tumor DNA analysis for mechanistically associated gene abnormalities and changes over time, and tumor analysis at baseline and on treatment using quantitative reverse transcriptase-polymerase chain reaction, ribonucleic acid sequencing, NanoString analysis and immunohistochemistry.

Relevant disease markers, such as tumor biomarkers, are collected at screening and during the study. At screening, all subjects provide a core or excisional biopsy obtained after the last dose of prior systemic therapy and before enrollment. Post-treatment biopsy of the same lesion is performed, if feasible, and if tumor persists in subjects who had a screening biopsy.

### Efficacy Assessment

RECIST 1.1 (Eisenhauer 2009) is used to assess tumor response, disease control rate (DCR), duration of response (DOR), and/or progression-free survival (PFS). The time of first study treatment administration to the first documented disease progression or death determines PFS. The time of first observed response to the first documented disease progression or death determines DOR.

Either computed CT or MRI is utilized to assess tumor response, with CT being the preferred imaging technique. All assessments at baseline and on study utilize the same methodology. All organs affected are documented as either target or non-target lesions at baseline per RECIST 1.1 and followed for the duration of the study.

A radiological assessment of a complete response (CR) or partial response (PR) requires confirmatory imaging at least 4 weeks after the initial assessment of response was observed. Significant clinical benefit may be used instead of a radiologically defined response to expand expansion cohorts.

### REFERENCES

American Cancer Society (ACS). Key Statistics for Ovarian Cancer. Web page accessed 16 Nov. 2022.
Bernal F. et al. Reactivation of the p53 Tumor Suppressor Pathway by a Stapled p53 Peptide. J. Am. Chem. Soc. 2007: 129:2456-2457.
Bird G H, et al. Biophysical Determinants for Cellular Uptake of Hydrocarbon-Stapled Peptide Helices. Nat. Chem. Biol. 2017; 12:845-852.
Bugter J M, et al. Mutations and mechanisms of WNT pathway tumour suppressors in cancer. Nat Rev Cancer. 2021 January; 21(1):5-21.
de la Roche M, et al. The function of BCL9 in Wnt/beta-catenin signaling and colorectal cancer cells. BMC Cancer. 2008 Jul. 15; 8:199.
Dotan E, et al. Phase Ib Study of Wnt Inhibitor Ipafricept with Gemcitabine and nab-paclitaxel in Patients with Previously Untreated Stage IV Pancreatic Cancer. Clin Cancer Res. 2020 Oct. 15; 26(20):5348-5357.
Dougherty P G, et al. Understanding Cell Penetration of Cyclic Peptides. Chem. Rev. 2019; 119(17):10241-10287.
Eisenhauer E A, et al. New response evaluation criteria in solid tumours: Revised RECIST guideline (version 1.1). Eur J Cancer 2009; 45(2):228-247.
Gay D M, et al. Loss of BCL9/91 suppresses Wnt driven tumourigenesis in models that recapitulate human cancer. Nat Commun. 2019 Feb. 13; 10(1):723.
Kahn M. Can we safely target the WNT pathway? Nat. Rev. Drug Discov. 2014 July; 13(7):513-532.
Lamarca A, et al. Second-line chemotherapy in advanced biliary cancer: a systematic review. Annals of oncology. 2014 Dec. 1; 25(12):2328-38. doi: 10.1093/annonc/mdul62.
Lin S Y, et al. Beta-catenin, a novel prognostic marker for breast cancer: its roles in cyclin D1 expression and cancer progression. Proc Natl Acad Sci USA. 2000 Apr. 11; 97(8):4262-6.
Liu J, et al. Wnt/β-catenin signalling: function, biological mechanisms, and therapeutic opportunities. Signal Transduct Target Ther. 2022 Jan. 3; 7(1):3.
Mieszczanek J. et al. Bcl9 and Pygo synergise downstream of Apc to effect intestinal neoplasia in FAP mouse models. Nat Commun. 2019 Feb. 13; 10(1):724.
Moor A E, et al. BCL9/9L-β-catenin Signaling is Associated With Poor Outcome in Colorectal Cancer. EBioMedicine. 2015 Oct. 30; 2(12):1932-43.
Moore K, et al. Endometrial Cancer: Is This a New Disease? Am. Soc. Clin. Oncol. Educational Book. 2017 435-442.
National Comprehensive Cancer Network (NCCN) Clinical Practice Guidelines in Oncology (NCCN guidelines) with NCCN Evidence Blocks—Ovarian Cancer 2022.
Nie X, et al. Emerging roles of Wnt ligands in human colorectal cancer. Frontiers in oncology. 2020 Aug. 14; 10:1341. doi: 10.3389/fonc.2020.01341
Parrish M L, et al. Mechanisms of mutant D-catenin in endometrial cancer progression. Frontiers Oncol. 2022 Sep. 29:12:1009345.
Pohl, S G, et al. Wnt signaling in triple-negative breast cancer. Oncogenesis. 2017; 6:e310. doi: 10.1038/oncsis.2017.14.
Ruiz de Galarreta M. et al. Q-Catenin Activation Promotes Immune Escape and Resistance to Anti-PD-1 Therapy in Hepatocellular Carcinoma. Cancer Discov 2019; 9(8):1124-41. doi: 10.1158/2159-8290.CD-19-0074.
Shang S, et al. The regulation of β-catenin activity and function in cancer: therapeutic opportunities. Oncotarget 2017; 8(20):33972-33989.
Shaw H V, et al. Targeting the Wnt signalling pathway in cancer: prospects and perils. Swiss Medical Weekly 2019; 149:w20129.
Simon R. Optimal two-stage designs for phase 2 clinical trials. Controlled Clinical Trials 1989:10:1-10.
Sung H, et al. Global Cancer Statistics 2020: GLOBOCAN Estimates of Incidence and Mortality Worldwide for 36 Cancers in 185 Countries. CA Cancer J Clin. 2021 May; 71(3):209-249. doi: 10.3322/caac.21660. Epub 2021 Feb. 4. PMID: 33538338.
Takada K, et al. Targeted disruption of the BCL9/β-catenin complex inhibits oncogenic Wnt signaling. Sci Transl Med. 2012 Aug. 22; 4(148).
Takahashi Y, et al. Surgical treatment of perihilar cholangiocarcinoma in octogenarians: a single center experience. J Hepatobiliary Pancreat Sci. 2013 March; 20(3):324-31.
Teeuwssen M, et al. Wnt signaling in ovarian cancer stemness, EMT, and therapy resistance. Journal of clinical medicine. 2019 Oct. 11; 8(10):1658.
Vafaizadeh V, et al. The interactions of Bcl9/Bcl9L with β-catenin and Pygopus promote breast cancer growth, invasion, and metastasis. Oncogene. 2021 October; 40(43):6195-6209.
Valle J, et al. Cisplatin plus gemcitabine versus gemcitabine for biliary tract cancer. N Engl J Med. 2010 Apr. 8; 362(14):1273-81. doi: 10.1056/NEJMoa0908721.
Wang J, et al. Low BCL9 expression inhibited ovarian epithelial malignant tumor progression by decreasing proliferation, migration, and increasing apoptosis to cancer cells. Cancer Cell Int. 2019 Dec. 6; 19:330.
Won K A, et al. Triple-negative breast cancer therapy: Current and future perspectives (Review). Int J Oncol. 2020 December; 57(6):1245-1261.
Zhang G F, et al. Wnt/β-catenin signaling as an emerging potential key pharmacological target in cholangiocarcinoma. Bioscience Reports. 2020 Mar. 27; 40(3).
Zhong Y, et al. Tankyrase Inhibition Causes Reversible Intestinal Toxicity in Mice with a Therapeutic Index<1. Toxicol Pathol. 2016 February; 44(2):267-78.

The present invention is further described by the following claims.

1. A method of treating a solid tumor in a patient, the method comprising parenterally administering to the patient a pharmaceutical composition comprising an effective amount of a peptide antagonist of β-catenin.

2. The method of claim 1, wherein the solid tumor is selected from the group consisting of breast cancer, colorectal cancer, cholangiocarcinoma, endometrial cancer, hepatocellular carcinoma, melanoma, non-small cell lung cancer, ovarian cancer, pancreatic adenocarcinoma, and synovial sarcoma.

3. The method of claim 1, wherein the patient has received a previous treatment selected from the group consisting of chemotherapy, hormone-based therapy, radiation, targeted therapy, and combinations thereof.

4. The method of claim 1, wherein the peptide antagonist is administered as a neoadjuvant.

5. The method of claim 1, wherein the peptide antagonist comprises the D-amino acid sequence FRWLLRQLARLAQLAKLTPVKLTPV (SEQ ID NO: 1).

6. The method of claim 1, wherein the peptide antagonist is administered to the patient at a dose of about 0.25-12 mg/kg.

7. A method of administering a peptide antagonist of β-catenin to a subject, the method comprising parenterally administering to the subject a pharmaceutical composition comprising the peptide antagonist,
wherein the peptide antagonist comprises the β-amino acid sequence FRWLLRQLARLAQLAKLTPVKLTPV (SEQ ID NO: 1), and
wherein the peptide antagonist is administered at a dose of about 0.25-12 mg/kg.

8. The method of claim 1, wherein the peptide antagonist is administered at a dose selected from about 0.5 mg/kg, about 1 mg/kg, about 2 mg/kg, about 4 mg/kg, about 8 mg/kg, and about 12 mg/kg.

9. The method of claim 1, wherein the peptide antagonist disrupts binding of β-catenin to B-cell CLL/lymphoma 9 protein (BCL9).

10. The method of claim 1, wherein the peptide antagonist comprises an N-terminal octanoyl group.

11. The method of claim 1, wherein the peptide antagonist is ST316.

12. The method of claim 1, wherein the pharmaceutical composition is administered intravenously.

13. The method or composition of claim 12, wherein the pharmaceutical composition is administered via infusion.

14. The method or composition of claim 13, wherein the infusion duration is about 30-180 minutes.

15. The method or composition of claim 14, wherein the infusion duration is about 60-90 minutes.

16. The method of claim 1, wherein the pharmaceutical composition is administered once weekly.

17. The method of claim 1, wherein the pharmaceutical composition is administered once every two weeks.

18. The method of claim 1, wherein the pharmaceutical composition is administered for at least four weeks.

19. The method of claim 1, further comprising administering to the patient bevacizumab and folinic acid, fluorouracil, and irinotecan (FOLFIRI).

20. The method of claim 1, further comprising administering to the patient fruquintinib.
